(19) United States
(12) Patent Application Publication
(10) Pub. No.: US 2025/0210171 A1
(43) Pub. Date: Jun. 26, 2025

(54) SYSTEMS AND METHODS FOR COMPLIANCE PREDICTION

(71) Applicant: Elevance Health, Inc., Indianapolis, IN (US)

(72) Inventors: Michael Johnston, Redding, CA (US); Mohit Singhal, Virginia Beach, VA (US); Hardik Patel, Granger, IN (US)

(21) Appl. No.: 18/986,671

(22) Filed: Dec. 18, 2024

Related U.S. Application Data
(60) Provisional application No. 63/613,102, filed on Dec. 21, 2023.

(51) Int. Cl. G16H 20/10; G06F 18/2415

(52) U.S. Cl. CPC G16H 20/10; G06F 18/2415

(57) ABSTRACT

Systems and methods of predicting regimen compliance and selecting electronic intervention plans are disclosed. A compliance event notification related to a regimen data structure is received and a next regimen compliance state for the regimen data structure is iteratively predicted for a prediction time period. The next regimen compliance state is predicted by a randomized prediction model including an integrated machine learning framework and the prediction time period is incremented by a predetermined increment during each iteration. When the next regimen compliance state comprises a compliant state, predicting at least one parameter of the next regimen compliance state by implementing a trained parameter prediction model comprising a tree-based machine learning framework. An intervention communication data structure is selected based on the next regimen compliance state and the regimen data structure is modified to reference the intervention communication data structure.

### RELATED APPLICATIONS

This application claims priority to U.S. Provisional Application No. 63/613,102, titled “Systems and Methods for Compliance Prediction,” filed Dec. 21, 2023, which is hereby incorporated by reference in its entirety.

### TECHNICAL FIELD

This application relates generally to predictive modeling, and more particularly, to predictive modeling for regimen compliance.

### BACKGROUND

Adherence to a regimen, such as regularly taking prescribed medications or regularly performing prescribed activities, is essential for regulatory compliance. With respect to healthcare regimens, adherence to a regimen is correlated with positive patient outcomes. Adherence to regimens may also be required for certain certifications, classifications, or benefits. For example, in order to obtain certain classification levels for a healthcare plan, a provider must demonstrate a minimum level of compliance for use of prescribed medications. In addition to meeting regulatory requirements, adherence to a regular schedules can provide for improved treatment and/or control of underlying issues.

In some instances, interventions, such as communications, can be used to increase compliance with regimens. For example, communications indicating upcoming events, such as prescription refills, or information regarding the importance of following regimens may increase adherence to a regimen. Although interventions can increase compliance, current systems do not provide processes or methods for identifying interventions based on compliance probabilities.

### SUMMARY

In various embodiments, a system is disclosed. The system includes a non-transitory memory and a processor communicatively coupled to the non-transitory memory. The processor is configured to read a set of instructions to receive a compliance event notification related to a regimen data structure and iteratively predict a next regimen compliance state for the regimen data structure for a prediction time period. The next regimen compliance state is predicted by a randomized prediction model including an integrated machine learning framework and the prediction time period is incremented by a predetermined increment during each iteration. The processor is further configured to read the set of instructions to select an intervention communication data structure based on the next regimen compliance state and modify the regimen data structure to reference the intervention communication data structure.

In various embodiments, a computer-implemented method is disclosed. The computer-implemented method includes steps of receiving a compliance event notification related to a regimen data structure and iteratively predicting a next regimen compliance state for the regimen data structure for a prediction time period. The next regimen compliance state is predicted by a randomized prediction model comprising a Monte Carlo framework including an integrated machine learning framework and the prediction time period is incremented by a predetermined increment during each iteration. The computer-implemented method further includes the steps of selecting an intervention communication data structure based on the next regimen compliance state and modifying the regimen data structure to reference the intervention communication data structure.

In various embodiments, a non-transitory computer readable medium having instructions stored thereon is disclosed. The instructions, when executed by at least one processor, cause at least one device to perform operations including receiving a compliance event notification related to a regimen data structure and iteratively predicting a next regimen compliance state for the regimen data structure for a prediction time period. The next regimen compliance state is predicted by a randomized prediction model including an integrated machine learning framework and the prediction time period is incremented by a predetermined increment during each iteration. The device is further configured to perform operations including classifying the regimen data structure in one of a plurality of categories based on the next regimen compliance state and a probability of the next regimen compliance state, selecting an intervention communication data structure based on the one of the plurality of categories, and modifying the regimen data structure to reference the intervention communication data structure.

### BRIEF DESCRIPTION OF THE DRAWINGS

The features and advantages of the present invention will be more fully disclosed in, or rendered obvious by the following detailed description of the preferred embodiments, which are to be considered together with the accompanying drawings wherein like numbers refer to like parts and further wherein:

FIG. 1 illustrates a network environment configured to provide automated compliance intervention, in accordance with some embodiments;

FIG. 2 illustrates a computer system configured to implement one or more processes, in accordance with some embodiments;

FIG. 3 is a flowchart illustrating an automated intervention generation method, in accordance with some embodiments;

FIG. 4 is a process flow illustrating various steps of the automated intervention generation method of FIG. 3, in accordance with some embodiments;

FIG. 5 is a partial system diagram illustrating various elements of a system configured to implement an automated intervention generation method of FIG. 3, in accordance with some embodiments;

FIG. 6 is graph illustrating a compliance trajectory for a corresponding regimen, in accordance with some embodiments;

FIG. 7 illustrates an artificial neural network, in accordance with some embodiments;

FIG. 8 illustrates a tree-based neural network, in accordance with some embodiments;

FIG. 9 illustrates a deep neural network (DNN), in accordance with some embodiments.

FIG. 10 is a flowchart illustrating a training method for generating a trained machine learning model, in accordance with some embodiments; and

FIG. 11 is a process flow illustrating various steps of the training method of FIG. 10, in accordance with some embodiments.

### DETAILED DESCRIPTION

This description of the exemplary embodiments is intended to be read in connection with the accompanying drawings, which are to be considered part of the entire written description. Terms concerning data connections, coupling and the like, such as “connected” and “interconnected,” and/or “in signal communication with” refer to a relationship wherein systems or elements are electrically connected (e.g., wired, wireless, etc.) to one another either directly or indirectly through intervening systems, unless expressly described otherwise. The term “operatively coupled” is such a coupling or connection that allows the pertinent structures to operate as intended by virtue of that relationship.

In the following, various embodiments are described with respect to the claimed systems as well as with respect to the claimed methods. Features, advantages, or alternative embodiments herein may be assigned to the other claimed objects and vice versa. In other words, claims for the systems may be improved with features described or claimed in the context of the methods. In this case, the functional features of the method are embodied by objective units of the systems. While the present disclosure is susceptible to various modifications and alternative forms, specific embodiments are shown by way of example in the drawings and will be described in detail herein. The objectives and advantages of the claimed subject matter will become more apparent from the following detailed description of these exemplary embodiments in connection with the accompanying drawings.

Furthermore, in the following, various embodiments are described with respect to methods and systems for automated compliance intervention. In various embodiments, a compliance event related to non-compliance of one or more regimen requirements for a predetermined regimen data structure are detected. In response to detection of the compliance event, a compliance state prediction model is configured to predict a next regimen compliance state for one or more prediction time periods. The compliance state prediction model is configured to iteratively predict a next regimen compliance state for subsequent time periods until a compliant state is predicted or a predetermined number of predictions have been executed. When a compliant state is predicted, a parameter prediction model predicts one or more parameters of the compliant state. The corresponding predetermined regimen data structure may be classified into one of a plurality of classes and an electronic communication intervention plan selected and/or implemented based on the classification. A prediction interface may be generated including interface elements configured to display the outcome of the predictions, the parameters of the predicted compliant state, elements of the selected electronic communication intervention plan, and/or any other suitable information.

In some embodiments, systems, and methods for automated compliance intervention include one or more trained compliance prediction models and/or parameter prediction models. The trained prediction models may include one or more models, such as trained randomized simulation models, trained extreme gradient boost models, trained tree models, etc.

In general, a trained function mimics cognitive functions that humans associate with other human minds. In particular, by training based on training data the trained function is able to adapt to new circumstances and to detect and extrapolate patterns.

In general, parameters of a trained function may be adapted by means of training. In particular, a combination of supervised training, semi-supervised training, unsupervised training, reinforcement learning and/or active learning may be used. Furthermore, representation learning (an alternative term is “feature learning”) may be used. In particular, the parameters of the trained functions may be adapted iteratively by several steps of training.

In some embodiments, a trained function may include a neural network, a support vector machine, a decision tree, a Bayesian network, a clustering network, Qlearning, genetic algorithms and/or association rules, and/or any other suitable artificial intelligence architecture. In some embodiments, a neural network may be a deep neural network, a convolutional neural network, a convolutional deep neural network, etc. Furthermore, a neural network may be an adversarial network, a deep adversarial network, a generative adversarial network, etc.

In various embodiments, neural networks which are trained (e.g., configured or adapted) to generate a predicted next regimen compliance state and/or parameters of a predicted state, are disclosed. A neural network trained to predict a next regimen compliance state may be referred to as a trained compliance prediction model and a neural network trained to predict parameters of a next regimen compliance state may be referred to as a trained parameter prediction model.

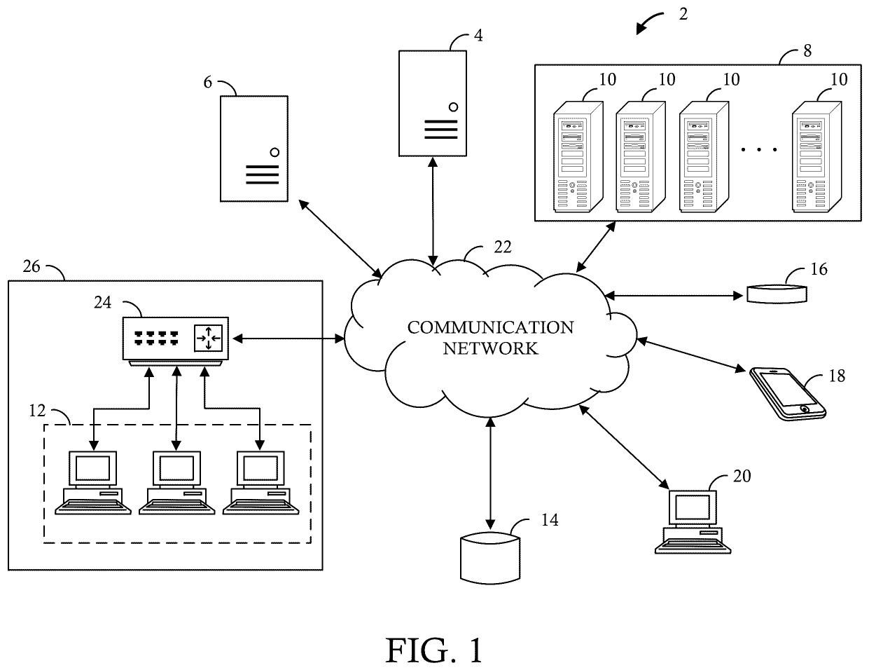

FIG. 1 illustrates a network environment 2 configured to provide automated compliance intervention, in accordance with some embodiments. The network environment 2 includes a plurality of devices or systems configured to communicate over one or more network channels, illustrated as a network cloud 22. For example, in various embodiments, the network environment 2 may include, but is not limited to, an intervention computing device 4, a web server 6, a cloud-based engine 8 including one or more processing devices 10, workstation(s) 12, a database 14, and/or one or more user computing devices 16, 18, 20 operatively coupled over the network 22. The intervention computing device 4, the web server 6, the processing device(s) 10, the workstation(s) 12, and/or the user computing devices 16, 18, 20 may each be a suitable computing device that includes any hardware or hardware and software combination for processing and handling information. For example, each computing device may include, but is not limited to, one or more processors, one or more field-programmable gate arrays (FPGAs), one or more application-specific integrated circuits (ASICs), one or more state machines, digital circuitry, and/or any other suitable circuitry. In addition, each computing device may transmit and receive data over the communication network 22.

In some embodiments, each of the intervention computing device 4 and the processing device(s) 10 may be a computer, a workstation, a laptop, a server such as a cloud-based server, or any other suitable device. In some embodiments, each of the processing devices 10 is a server that includes one or more processing units, such as one or more graphical processing units (GPUs), one or more central processing units (CPUs), and/or one or more processing cores. Each processing device 10 may, in some embodiments, execute one or more virtual machines. In some embodiments, processing resources (e.g., capabilities) of the one or more processing devices 10 are offered as a cloud-based service (e.g., cloud computing). For example, the cloud-based engine 8 may offer computing and storage resources of the one or more processing devices 10 to the intervention computing device 4.

In some embodiments, each of the user computing devices 16, 18, 20 may be a cellular phone, a smart phone, a tablet, a personal assistant device, a voice assistant device, a digital assistant, a laptop, a computer, or any other suitable device. In some embodiments, the web server 6 hosts one or more network environments, such as an e-commerce network environment. In some embodiments, the intervention computing device 4, the processing devices 10, and/or the web server 6 are operated by the network environment provider, and the user computing devices 16, 18, 20 are operated by users of the network environment. In some embodiments, the processing devices 10 are operated by a third party (e.g., a cloud-computing provider).

The workstation(s) 12 are operably coupled to the communication network 22 via a router (or switch) 24. The workstation(s) 12 and/or the router 24 may be located at a physical location 26 remote from the intervention computing device 4, for example. The workstation(s) 12 may communicate with the intervention computing device 4 over the communication network 22. The workstation(s) 12 may send data to, and receive data from, the intervention computing device 4. For example, the workstation(s) 12 may transmit data related to tracked operations performed at the physical location 26 to intervention computing device 4.

Although FIG. 1 illustrates three user computing devices 16, 18, 20, the network environment 2 may include any number of user computing devices 16, 18, 20. Similarly, the network environment 2 may include any number of the intervention computing device 4, the web server 6, the processing devices 10, the workstation(s) 12, and/or the databases 14. It will further be appreciated that additional systems, servers, storage mechanism, etc. may be included within the network environment 2. In addition, although embodiments are illustrated herein having individual, discrete systems, it will be appreciated that, in some embodiments, one or more systems may be combined into a single logical and/or physical system. For example, in various embodiments, one or more of the intervention computing device 4, the web server 6, the workstation(s) 12, the database 14, the user computing devices 16, 18, 20, and/or the router 24 may be combined into a single logical and/or physical system. Similarly, although embodiments are illustrated having a single instance of each device or system, it will be appreciated that additional instances of a device may be implemented within the network environment 2. In some embodiments, two or more systems may be operated on shared hardware in which each system operates as a separate, discrete system utilizing the shared hardware, for example, according to one or more virtualization schemes.

The communication network 22 may be a WiFi® network, a cellular network such as a 3GPP® network, a Bluetooth® network, a satellite network, a wireless local area network (LAN), a network utilizing radio-frequency (RF) communication protocols, a Near Field Communication (NFC) network, a wireless Metropolitan Area Network (MAN) connecting multiple wireless LANs, a wide area network (WAN), or any other suitable network. The communication network 22 may provide access to, for example, the Internet.

Each of the first user computing device 16, the second user computing device 18, and the Nth user computing device 20 may communicate with the web server 6 over the communication network 22. For example, each of the user computing devices 16, 18, 20 may be operable to view, access, and interact with a website, such as an e-commerce website, hosted by the web server 6. The web server 6 may transmit user session data related to a user's activity (e.g., interactions) on the website. For example, a user may operate one of the user computing devices 16, 18, 20 to initiate a web browser that is directed to the website hosted by the web server 6. The user may, via the web browser, perform various operations such as searching one or more databases or catalogs associated with the displayed website, view item data for elements associated with and displayed on the website, and click on interface elements presented via the website, for example, in the search results. The website may capture these activities as user session data, and transmit the user session data to the intervention computing device 4 over the communication network 22. The website may also allow the user to interact with one or more of interface elements to perform specific operations, such as selecting one or more items for further processing. In some embodiments, the web server 6 transmits user interaction data identifying interactions between the user and the website to the intervention computing device 4.

In some embodiments, the intervention computing device 4 may execute one or more models, processes, or algorithms, such as a machine learning model, deep learning model, statistical model, etc., to predict a next regimen compliance state and/or parameters of a next regimen compliance state. The intervention computing device 4 may transmit electronic intervention messages to the web server 6 over the communication network 22, and the web server 6 may display interface elements associated with electronic intervention messages on the website to the user. For example, the web server 6 may display interface elements associated with electronic intervention messages to the user on a homepage.

The intervention computing device 4 is further operable to communicate with the database 14 over the communication network 22. For example, the intervention computing device 4 may store data to, and read data from, the database 14. The database 14 may be a remote storage device, such as a cloud-based server, a disk (e.g., a hard disk), a memory device on another application server, a networked computer, or any other suitable remote storage. Although shown remote to the intervention computing device 4, in some embodiments, the database 14 may be a local storage device, such as a hard drive, a non-volatile memory, or a USB stick. The intervention computing device 4 may store interaction data received from the web server 6 in the database 14. The intervention computing device 4 may also receive from the web server 6 user session data identifying events associated with browsing sessions, and may store the user session data in the database 14.

In some embodiments, the intervention computing device 4 generates training data for a plurality of models (e.g., machine learning models, deep learning models, statistical models, algorithms, etc.) based on aggregation data, variant-level data, holiday and event data, recall data, historical user session data, search data, purchase data, catalog data, advertisement data for the users, etc. The intervention computing device 4 and/or one or more of the processing devices 10 may train one or more models based on corresponding training data. The intervention computing device 4 may store the models in a database, such as in the database 14 (e.g., a cloud storage database).

The models, when executed by the intervention computing device 4, allow the intervention computing device 4 to predict a next regimen compliance state for a compliance object and/or parameters of a predicted next regimen compliance state. For example, the intervention computing device 4 may obtain one or more models from the database 14. The intervention computing device 4 may execute one or more models to predict a next regimen compliance state in response to a detected compliance event and/or predict parameters of a predicted next regimen compliance state.

In some embodiments, the intervention computing device 4 assigns the models (or parts thereof) for execution to one or more processing devices 10. For example, each model may be assigned to a virtual machine hosted by a processing device 10. The virtual machine may cause the models or parts thereof to execute on one or more processing units such as GPUs. In some embodiments, the virtual machines assign each model (or part thereof) among a plurality of processing units. Based on the output of the models, intervention computing device 4 may automatically generate an electronic communication intervention plan and/or electronic communications based on an electronic communication intervention plan.

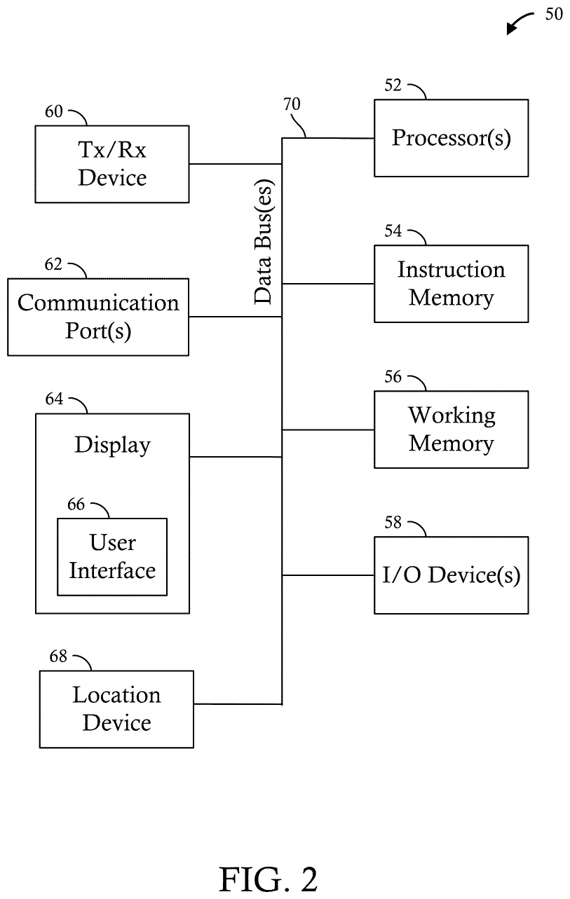

FIG. 2 illustrates a block diagram of a computing device 50, in accordance with some embodiments. In some embodiments, each of the intervention computing device 4, the web server 6, the one or more processing devices 10, the workstation(s) 12, and/or the user computing devices 16, 18, 20 in FIG. 1 may include the features shown in FIG. 2. Although FIG. 2 is described with respect to certain components shown therein, it will be appreciated that the elements of the computing device 50 may be combined, omitted, and/or replicated. In addition, it will be appreciated that additional elements other than those illustrated in FIG. 2 may be added to the computing device.

As shown in FIG. 2, the computing device 50 may include one or more processors 52, an instruction memory 54, a working memory 56, one or more input/output devices 58, a transceiver 60, one or more communication ports 62, a display 64 with a user interface 66, and an optional location device 68, all operatively coupled to one or more data buses 70. The data buses 70 allow for communication among the various components. The data buses 70 may include wired, or wireless, communication channels.

The one or more processors 52 may include any processing circuitry operable to control operations of the computing device 50. In some embodiments, the one or more processors 52 include one or more distinct processors, each having one or more cores (e.g., processing circuits). Each of the distinct processors may have the same or different structure. The one or more processors 52 may include one or more central processing units (CPUs), one or more graphics processing units (GPUs), application specific integrated circuits (ASICs), digital signal processors (DSPs), a chip multiprocessor (CMP), a network processor, an input/output (I/O) processor, a media access control (MAC) processor, a radio baseband processor, a co-processor, a microprocessor such as a complex instruction set computer (CISC) microprocessor, a reduced instruction set computing (RISC) microprocessor, and/or a very long instruction word (VLIW) microprocessor, or other processing device. The one or more processors 52 may also be implemented by a controller, a microcontroller, an application specific integrated circuit (ASIC), a field programmable gate array (FPGA), a programmable logic device (PLD), etc.

In some embodiments, the one or more processors 52 are configured to implement an operating system (OS) and/or various applications. Examples of an OS include, for example, operating systems generally known under various trade names such as Apple macOS™, Microsoft Windows™, Android™, Linux™, and/or any other proprietary or open-source OS. Examples of applications include, for example, network applications, local applications, data input/output applications, user interaction applications, etc.

The instruction memory 54 may store instructions that are accessed (e.g., read) and executed by at least one of the one or more processors 52. For example, the instruction memory 54 may be a non-transitory, computer-readable storage medium such as a read-only memory (ROM), an electrically erasable programmable read-only memory (EEPROM), flash memory (e.g. NOR and/or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, a removable disk, CD-ROM, any non-volatile memory, or any other suitable memory. The one or more processors 52 may be configured to perform a certain function or operation by executing code, stored on the instruction memory 54, embodying the function or operation. For example, the one or more processors 52 may be configured to execute code stored in the instruction memory 54 to perform one or more of any function, method, or operation disclosed herein.

Additionally, the one or more processors 52 may store data to, and read data from, the working memory 56. For example, the one or more processors 52 may store a working set of instructions to the working memory 56, such as instructions loaded from the instruction memory 54. The one or more processors 52 may also use the working memory 56 to store dynamic data created during one or more operations. The working memory 56 may include, for example, random access memory (RAM) such as a static random access memory (SRAM) or dynamic random access memory (DRAM), Double-Data-Rate DRAM (DDR-RAM), synchronous DRAM (SDRAM), an EEPROM, flash memory (e.g. NOR and/or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, a removable disk, CD-ROM, any non-volatile memory, or any other suitable memory. Although embodiments are illustrated herein including separate instruction memory 54 and working memory 56, it will be appreciated that the computing device 50 may include a single memory unit configured to operate as both instruction memory and working memory. Further, although embodiments are discussed herein including non-volatile memory, it will be appreciated that computing device 50 may include volatile memory components in addition to at least one non-volatile memory component.

In some embodiments, the instruction memory 54 and/or the working memory 56 includes an instruction set, in the form of a file for executing various methods, such as methods for automated compliance intervention, as described herein. The instruction set may be stored in any acceptable form of machine-readable instructions, including source code or various appropriate programming languages. Some examples of programming languages that may be used to store the instruction set include, but are not limited to: Java, JavaScript, C, C++, C#, Python, Objective-C, Visual Basic, .NET, HTML, CSS, SQL, NoSQL, Rust, Perl, etc. In some embodiments a compiler or interpreter is configured to convert the instruction set into machine executable code for execution by the one or more processors 52.

The input-output devices 58 may include any suitable device that allows for data input or output. For example, the input-output devices 58 may include one or more of a keyboard, a touchpad, a mouse, a stylus, a touchscreen, a physical button, a speaker, a microphone, a keypad, a click wheel, a motion sensor, a camera, and/or any other suitable input or output device.

The transceiver 60 and/or the communication port(s) 62 allow for communication with a network, such as the communication network 22 of FIG. 1. For example, if the communication network 22 of FIG. 1 is a cellular network, the transceiver 60 is configured to allow communications with the cellular network. In some embodiments, the transceiver 60 is selected based on the type of the communication network 22 the computing device 50 will be operating in. The one or more processors 52 are operable to receive data from, or send data to, a network, such as the communication network 22 of FIG. 1, via the transceiver 60.

The communication port(s) 62 may include any suitable hardware, software, and/or combination of hardware and software that is capable of coupling the computing device 50 to one or more networks and/or additional devices. The communication port(s) 62 may be arranged to operate with any suitable technique for controlling information signals using a desired set of communications protocols, services, or operating procedures. The communication port(s) 62 may include the appropriate physical connectors to connect with a corresponding communications medium, whether wired or wireless, for example, a serial port such as a universal asynchronous receiver/transmitter (UART) connection, a Universal Serial Bus (USB) connection, or any other suitable communication port or connection. In some embodiments, the communication port(s) 62 allows for the programming of executable instructions in the instruction memory 54. In some embodiments, the communication port(s) 62 allow for the transfer (e.g., uploading or downloading) of data, such as machine learning model training data.

In some embodiments, the communication port(s) 62 are configured to couple the computing device 50 to a network. The network may include local area networks (LAN) as well as wide area networks (WAN) including without limitation Internet, wired channels, wireless channels, communication devices including telephones, computers, wire, radio, optical and/or other electromagnetic channels, and combinations thereof, including other devices and/or components capable of/associated with communicating data. For example, the communication environments may include in-body communications, various devices, and various modes of communications such as wireless communications, wired communications, and combinations of the same.

In some embodiments, the transceiver 60 and/or the communication port(s) 62 are configured to utilize one or more communication protocols. Examples of wired protocols may include, but are not limited to, Universal Serial Bus (USB) communication, RS-232, RS-422, RS-423, RS-485 serial protocols, FireWire, Ethernet, Fibre Channel, MIDI, ATA, Serial ATA, PCI Express, T-1 (and variants), Industry Standard Architecture (ISA) parallel communication, Small Computer System Interface (SCSI) communication, or Peripheral Component Interconnect (PCI) communication, etc. Examples of wireless protocols may include, but are not limited to, the Institute of Electrical and Electronics Engineers (IEEE) 802.xx series of protocols, such as IEEE 802.11a/b/g/n/ac/ag/ax/be, IEEE 802.16, IEEE 802.20, GSM cellular radiotelephone system protocols with GPRS, CDMA cellular radiotelephone communication systems with 1×RTT, EDGE systems, EV-DO systems, EV-DV systems, HSDPA systems, Wi-Fi Legacy, Wi-Fi 1/2/3/4/5/6/6E, wireless personal area network (PAN) protocols, Bluetooth Specification versions 5.0, 6, 7, legacy Bluetooth protocols, passive or active radio-frequency identification (RFID) protocols, Ultra-Wide Band (UWB), Digital Office (DO), Digital Home, Trusted Platform Module (TPM), ZigBec, etc.

The display 64 may be any suitable display, and may display the user interface 66. The user interfaces 66 may enable user interaction with model predictions, such as next regimen compliance state predictions and/or parameters of next regimen compliance state predictions. For example, the user interface 66 may be a user interface for an application of a network environment operator that allows a user to view and interact with the operator's website. In some embodiments, a user may interact with the user interface 66 by engaging the input-output devices 58. In some embodiments, the display 64 may be a touchscreen, where the user interface 66 is displayed on the touchscreen.

The display 64 may include a screen such as, for example, a Liquid Crystal Display (LCD) screen, a light-emitting diode (LED) screen, an organic LED (OLED) screen, a movable display, a projection, etc. In some embodiments, the display 64 may include a coder/decoder, also known as Codecs, to convert digital media data into analog signals. For example, the visual peripheral output device may include video Codecs, audio Codecs, or any other suitable type of Codec.

The optional location device 68 may be communicatively coupled to the a location network and operable to receive position data from the location network. For example, in some embodiments, the location device 68 includes a GPS device configured to receive position data identifying a latitude and longitude from one or more satellites of a GPS constellation. As another example, in some embodiments, the location device 68 is a cellular device configured to receive location data from one or more localized cellular towers. Based on the position data, the computing device 50 may determine a local geographical area (e.g., town, city, state, etc.) of its position.

In some embodiments, the computing device 50 is configured to implement one or more modules or engines, each of which is constructed, programmed, configured, or otherwise adapted, to autonomously carry out a function or set of functions. A module/engine may include a component or arrangement of components implemented using hardware, such as by an application specific integrated circuit (ASIC) or field-programmable gate array (FPGA), for example, or as a combination of hardware and software, such as by a microprocessor system and a set of program instructions that adapt the module/engine to implement the particular functionality, which (while being executed) transform the microprocessor system into a special-purpose device. A module/engine may also be implemented as a combination of the two, with certain functions facilitated by hardware alone, and other functions facilitated by a combination of hardware and software. In certain implementations, at least a portion, and in some cases, all, of a module/engine may be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each module/engine may be realized in a variety of physically realizable configurations, and should generally not be limited to any particular implementation exemplified herein, unless such limitations are expressly called out. In addition, a module/engine may itself be composed of more than one sub-modules or sub-engines, each of which may be regarded as a module/engine in its own right. Moreover, in the embodiments described herein, each of the various modules/engines corresponds to a defined autonomous functionality; however, it should be understood that in other contemplated embodiments, each functionality may be distributed to more than one module/engine. Likewise, in other contemplated embodiments, multiple defined functionalities may be implemented by a single module/engine that performs those multiple functions, possibly alongside other functions, or distributed differently among a set of modules/engines than specifically illustrated in the embodiments herein.

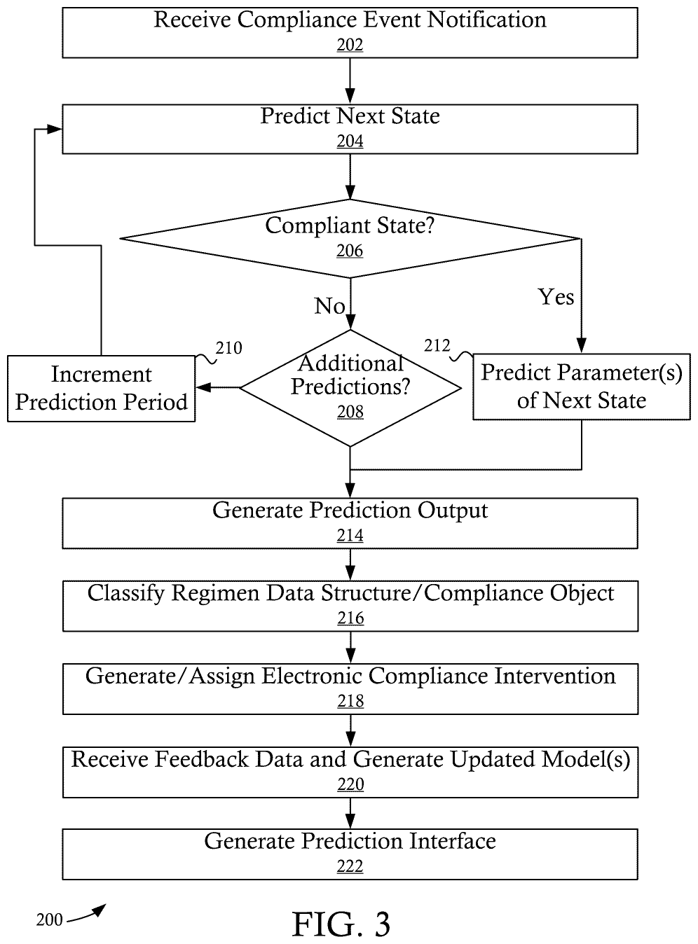

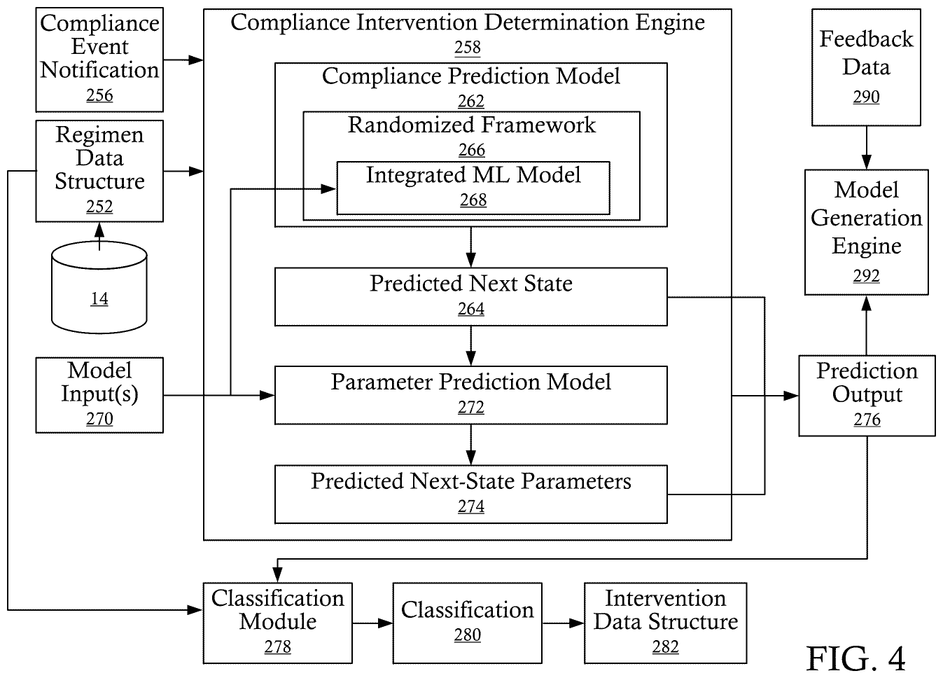

FIG. 3 is a flowchart illustrating an automated intervention generation method 200, in accordance with some embodiments. FIG. 4 is a process flow 250 illustrating various steps of the automated intervention generation method 200, in accordance with some embodiments. In some embodiments, the automated intervention generation method 200 is configured to automatically generate electronic communications and/or electronic communication plans based on a likelihood of non-compliance with a predetermined regimen. In some embodiments, the automated intervention generation method 200 is configured to generate an electronic communication plan for predetermined regimen data structures 252.

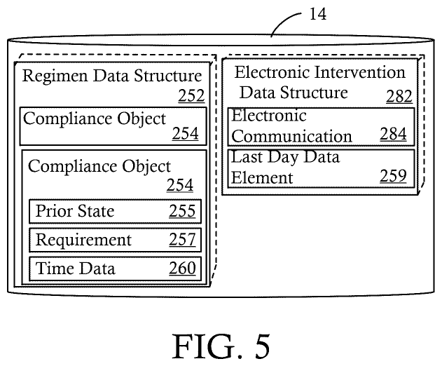

FIG. 5 illustrates one embodiment of a database 14 including a predetermined regimen data structure 252. In some embodiments, a predetermined regimen data structure 252 is configured to store data representative of one or more tracked and/or expected elements of a predetermined regimen. A predetermined regimen data structure 252 may include one or more compliance objects 254 representative of one or more compliance requirements of the predetermined regimen. As one non-limiting example, with respect to predetermined regimen data structures 252 related to healthcare regimens, compliance requirements may include, but are not limited to, medication schedules, office visit schedules, biometric recording schedules, etc. As another non-limiting example, with respect to predetermined regimen data structures 252 related to maintenance regimens, compliance requirements may include, but are not limited to, maintenance schedules, check-in schedules, obtained measurements or readings, etc.

With reference to FIGS. 3-5, at step 202, a compliance event notification 256 is received. The compliance event notification 256 may be received by any suitable system, method, module, engine, etc., such as, for example, a compliance intervention determination engine 258. The compliance event notification 256 includes data indicating occurrence of an event related to a predetermined regimen data structure 252. In some embodiments, the compliance event notification 256 is related to one or more compliance objects 254 defined by the predetermined regimen data structure 252. In some embodiments, each of the compliance objects 254 is representative of a portion of the predetermined regimen data structure 252 requiring on-going and/or periodic compliance and a compliance event notification 256 may be related to compliance with a compliance object 254. As one non-limiting example, in embodiments including a predetermined regimen data structure 252 including a compliance object 254 representative of a medication regimen for a prescription medication, a compliance event notification 256 may be related to compliance with the medication regimen. As another non-limiting example, in embodiments including a predetermined regimen data structure 252 including a compliance object 254 representative of a maintenance regimen, a compliance event notification 256 may be related to compliance with a maintenance schedule.

In some embodiments, a compliance event notification 256 may include an exhaustion event for one or more compliance objects 254. An exhaustion event may occur when a compliance period (e.g., a predetermined time period) has elapsed. The compliance period may be related to a predetermined time period for a provided quantity of an element (e.g., medication), such as a period during which the provided element should have been consumed according to the predetermined regimen. An exhaustion event notification may be generated when the compliance period has elapsed and/or expired. In some embodiments, an exhaustion event occurs when the predetermined time period has elapsed without an additional event occurring, such as a refill of the one or more elements being obtained.

In some embodiments, a compliance object 254 related to an event having a predetermined time period may include a time data element 260. The time data element 260 may be indicative of the start of a compliance period, a date on which a compliance period expires, data for determining a compliance period, and/or any other suitable data for tracking and/or determining a compliance period. The time data element 260 may include any suitable format and/or granularity related to the compliance period, such as, for example, providing day, hour, minute, etc. granularity. Although embodiments are discussed herein including an exhaustion event, it will be appreciated that any suitable compliance notification event 256 may be received. Examples of other compliance event notifications 256 may include, but are not limited to, missed appointments, failure to complete assigned tasks, incomplete or missing information, etc.

As one non-limiting example, in some embodiments, a compliance object 254 may define a prescription regimen requiring consumption of a provided medication as part of a predetermined healthcare regimen. The compliance object 254 may include data elements defining a specific dosage of the medication and a predetermined time interval for the dosage. When a prescription fill event is received, the compliance object 254 is updated to include a time data element 260 indicating an expected date on which the predetermined amount of the medication will be consumed if the medication regimen is followed correctly, e.g., a compliance period for the prescription fill event. A compliance object related to a prescription regimen may have any suitable compliance period, such as, for example, 30 days from a fill event, 60 days from a fill event, 90 days from a fill event, etc. When the compliance period elapses, an exhaustion event notification may be generated indicating the compliance period has expired without a subsequent fill event (e.g., a refill event) being detected or recorded. Although embodiments are discussed herein including a compliance object 254 related to a prescription regimen, it will be appreciated that the compliance object 254 may track any suitable regimen or sub-regimen and have any suitable related compliance events, notifications, and/or compliance periods.

In some embodiments, a compliance event notification 256 may be generated by a simulated compliance event. For example, in some embodiments, a compliance event notification 256 may be generated to simulate the occurrence of a compliance event for a selected predetermined regimen data structure 252. Simulated compliance events may be used to predict prior and/or future compliance for one or more compliance objects 254 and, as discussed in greater detail below, generate electronic communications configured to increase a probability of compliance for the compliance object 254 during a time period subsequent to the simulated compliance event.

At step 204, a next regimen compliance state 264 is predicted for the compliance object 254 associated with the compliance event notification 256. The next regimen compliance state 264 includes a most-probable (e.g., most-likely) one of a plurality of potential compliance states of the compliance object 254 at the end of a prediction time period. In some embodiments, a next regimen compliance state 264 may include a binary prediction indicating one of two potential compliance states, e.g., compliant or non-compliant. The prediction time period may include any suitable time period related to the compliance object 254, such as, for example, a selected day, week, month, etc.

The next regimen compliance state 264 may be determined based on a probability (e.g., likelihood) that the event underlying the compliance event notification 256 will be addressed (e.g., remedied, corrected, etc.) during the prediction time period. For example, in the context of a compliance object 254 related to a medication regimen, a next regimen compliance state 264 may include a compliant state predicting a prescription refill will be obtained by and/or on the prediction time period and a non-compliant state predicting that a prescription refill will not be obtained by and/or on the prediction time period. As another example, in the context of a compliance object 254 related to a treatment regimen, a next regimen compliance state 264 may be include a compliant state predicting a prescribed treatment will occur by and/or on the prediction time period and a non-compliant state predicting that a treatment will not occur by and/or on the prediction time period.

In some embodiments, the next regimen compliance state 264 is predicted, at least in part, by a compliance prediction model 262. In some embodiments, the compliance prediction model 262 includes a randomized simulation framework 266 including an integrated machine learning framework 268. The integrated machine learning framework 268 may be configured to generate one or more parameters and/or outputs required by and/or used by the randomized simulation framework. The randomized simulation framework 266 may include, but is not limited to, a Monte Carlo framework, a Markoff Chain framework, a Monte Carlo Markoff Chain (MCMC) framework, etc. The integrated machine learning framework 268 may include, but is not limited to, a trained gradient boosting model such as extreme gradient boost (XGB), a trained tree-based model such as random forest and/or decision tree models, and/or any other suitable machine learning model. In some embodiments, the compliance prediction model 262 includes a Monte Carlo framework incorporating an integrated XGB model. Although specific embodiments are discussed herein, it will be appreciated that any suitable combination of randomized simulation framework and/or machine learning model may be implemented by the compliance prediction model 262.

In some embodiments, the randomized simulation framework 266 generates a next regimen compliance state 264 as a composite prediction. For example, a randomized simulation framework may be configured to perform multiple simulations, each configured to generate a simulation-specific predicted state. The randomized simulation framework may be configured to execute a predetermined number of simulations and/or may be configured to execute a variable number of simulations based on one or more parameters, such as a confidence threshold. In some embodiments, the next regimen compliance state 264 may be predicted by aggregating the simulation-specific predictions to determine a most-likely future state. For example, if a number of simulations that predict a first state is above a first threshold (e.g., the number of simulations predicting the first state is more than half of the executed simulations), the randomized simulation framework generates an aggregated next regimen compliance state 264 predicting a first state. In some embodiments, a confidence interval is applied to simulation-specific predictions prior to and/or in conjunction with generation of the next regimen compliance state 264.

In some embodiments, the compliance prediction model 262 is configured to implement two or more randomized simulations in parallel and/or series. For example, in some embodiments, multiple instances of the randomized simulation framework may be executed in parallel (e.g., buy multiple processors and/or processor cores) with each instance including an integrated machine learning framework 268 configured to generate a next regimen compliance state prediction. Each of the instances of the randomized simulation framework may vary one or more inputs and/or parameters of the simulation and/or the integrated machine learning framework 268 based on the randomized framework. The outputs of each of the instances of the randomized simulation framework may be integrated to generate an aggregated predicted next regimen compliance state 264.

In some embodiments, the integrated machine learning framework 268 is configured to receive a set of inputs 270 and predict a next regimen compliance state 264 for a specific time period. The set of inputs 270 may include one or more parameters or features related to a field of the prediction, the compliance event notification 256, and/or any other suitable input. For example, the set of inputs 270 may include, but is not limited to, compliance-related features, demographic-related features, domain-specific features, product-related features, event-related features, etc. The set of inputs 270 may include one or more inputs selected prior to model training and/or one or more inputs selected iteratively during a model training process, as discussed in greater detail below. The compliance-related features may include features representative of and/or related to prior compliance related to the compliance object 254 and/or the compliance event notification 256. For example, in embodiments including a compliance object 254 related to a medication regimen, the compliance-related features may include, but are not limited to, features representative of a one or more elements of a prescribed medication, prior compliance data for predetermined time periods (e.g., prior month, prior year, etc.), elements of additionally prescribed medications, number of refills for a prescribed medication in a prior time period, gaps between prescription fills, gap periods between prescription fills, number of days since the compliance event occurred, a specific day within a predetermined time period (e.g., day of the year), number of fills within a predetermined time period, proportion of a predetermined time period during which compliance object 254 was in a compliant state, etc.

In some embodiments, the set of demographic-related features may include features related to an individual, e.g., patient, associated with the compliance event and/or the predetermined regimen. For example, the demographic related features may include, but are not limited to, age, gender, primary language, etc. Demographic-related features may include individual-specific features, anonymized demographic features, and/or generalized demographic features. In some embodiments, the domain-specific features may include features selected for a domain related to the compliance event notification 256. For example, in embodiments including a compliance object 254 related to a medication regimen and a related compliance event notification 256, such as a compliance notification related to an expiration event, the domain-specific features may include, but are not limited to, social determinations of health (SDoH)-related features, product or provider-related features, etc.

In some embodiments, the product-related features may include features related to provision of a product. For example, in embodiments including a prescription adherence prediction, product related features may include, but are not limited to, enrollment in one or more product related programs (e.g., loyalty programs, monitoring programs, etc.), programs or plans through which the prescription is being provided (e.g., plan type, plan specifics), etc. In some embodiments, event-related features may include features related to the triggering compliance event, e.g., the compliance event embodied in the compliance event notification 256. For example, in embodiments including a prescription adherence prediction, event-related features may include, but are not limited to, date of a prior prescription refill, the number of days in the prior prescription refill, etc. Although specific embodiments are discussed herein, it will be appreciated that any suitable set of features may be received by a compliance prediction model 262.

In some embodiments, the set of inputs 270 includes one or more inputs defined by prior iterations of automated intervention generation method 200 and/or a sub-portion thereof. For example, one or more electronic intervention messages may be generated and transmitted to a user device associated with a predetermined regimen data structure 252, as discussed in greater detail below. One or more inputs may be representative of electronic intervention messages and/or responses to electronic intervention messages generated during prior time periods and/or prior iterations of the automated intervention generation method 200. Although specific embodiments are discussed herein, it will be appreciated that the set of inputs 270 may be expanded and/or contracted to utilize any suitable set of inputs defined prior to, during, and/or after iterative training of one or more machine learning frameworks.

In some embodiments, the integrated machine learning framework is configured to receive one or more features and/or feature values from one or more data sources, such as one or more databases. The data sources may include data systems configured to track and/or maintain data related to events other than a compliance event, such as, for example, a fulfillment system, a claims system, etc. In some embodiments, the data sources may include user, e.g., patient, inputs and/or patient-application tracked features.

At step 206, a determination is made whether the predicted next regimen compliance state 264 indicates compliance with the compliance object 254 (e.g., indicates correction of the compliance event related to the compliance event notification 256). When the predicted next regimen compliance state 262 is in compliance with the compliance object 254, the method 200 proceeds to step 210 and at least one parameter of the next regimen compliance state 264 is determined. Alternatively, when the next regimen compliance state 264 is not in compliance (e.g., the compliance event has not been corrected), the prediction time period is updated (e.g., incremented) and the method 200 returns to step 204 to iteratively predict a next regimen compliance state 264 for the updated prediction time period. For example, an initial prediction time period may include a first day (e.g., Day₀) and the prediction time period may be incremented by a day such that subsequent iterations of step 204 predict a next regimen compliance state 264 for subsequent days (e.g., Day₁, Day₂, etc.). Although specific embodiments are discussed herein, it will be appreciated that the initial time period may include any suitable time period and the prediction time period may be incremented by any suitable increment, such as, for example, one or more days, hours, minutes, etc.

In some embodiments, the first integrated machine learning framework 268 is configured to generate a probability that the event related to the compliance event notification 256 is remedied within the prediction time period. As one non-limiting example, in embodiments including a compliance object 254 representative of a medication regimen, a next regimen compliance state 264 is selected based on a prediction that a refill of a corresponding prescription is obtained (e.g., a compliant state) or that a refill is not obtained (e.g., a non-compliant state) during the selected prediction time period. In some embodiments, the compliance prediction model 262 and the potential next compliant state(s) 264 are selected based on and/or defined by the compliance object 254.

At optional step 208, a determination is made whether a subsequent time period prediction is required. When the determination at step 208 indicates a subsequent prediction is required, the method 200 proceeds to step 210, the prediction period is incremented, and the method 200 iteratively repeats steps 204 and 206. Alternatively, when the determination at step 208 indicates a subsequent prediction is not required, the method 200 proceeds to step 212. For example, in some embodiment, the method 200 may iteratively repeat steps 204 and 206 until the predicted next regimen compliance state 264 is a compliant state or until a predetermined number of predictions have been generated. When the next regimen compliance state 264 predicted at step 204 during an initial time period (e.g., Day₀) is a non-compliant state, the prediction time period may be incremented by one day and the compliance prediction model 262 predicts a next regimen compliance state 264 for the subsequent time period (e.g., Day₁). In some embodiments, the compliance prediction model 262 implements the randomized simulation framework 266 to generate a new composite prediction based on the integrated machine learning framework 268. The integrated machine learning framework 268 receives an updated input set 270 including at least one updated input (e.g., prediction time period). It will be appreciated that additional inputs may also be updated, such as inputs related to a time elapsed since a prior compliance event and/or predetermined time period. The prediction time period may be iteratively incremented until a compliant state is predicted and/or a predetermined cutoff is reached.

In some embodiments, the method 200 is configured to iteratively repeat steps 204-210 until a final prediction time period is reached. To continue the example from above, if the compliance prediction model 262 predicts a next regimen compliance state 264 for the updated time period Day₁, the method 200 iteratively increments the prediction time period until a compliant state has been predicted or a final prediction time period (e.g., Day_{final}) is reached. The prediction time period may be incremented over any suitable length of time. For example, in some embodiments including a prediction time period including daily increment (e.g., Day₀, Day₁, . . . , Day_{final}), the time period from Day₀ to Day_{final} may include any suitable time period, such as one month, three months, six months, etc. In some embodiments, the compliance prediction model 262 is configured to generate an output indicative of the prediction time period for which compliance was predicted and/or an output indicating no predicted compliant states during the prediction period (e.g., from Day₀ to Day_{final}).

In some embodiments, the method 200 is configured to iteratively repeat steps 204-210 until a predetermined number of non-compliant states are predicted in a row. For example, in some embodiments, when the compliance prediction model 262 predicts a next regimen compliance state 264 that is non-compliant, a counter may be incremented indicating the current number of predicted non-compliant states. If the value of the counter is incremented to be equal to or greater than a predetermined threshold value prior to a compliant next regimen compliance state 264 being predicted, the determination at step 208 may indicate that additional predictions are unnecessary, for example, as a compliant state may be unlikely to occur after a certain number of non-compliant states and/or may no longer matter. Although specific embodiments are discussed herein, it will be appreciated that any suitable determination may be made at step 208 to determine whether additional predictions are required.

As discussed above, when the compliance prediction model 262 predicts a compliant next regimen compliance state 264, the method 200 proceeds to step 212. At step 212, one or more parameters 274 of the next regimen compliance state 264 of the compliance object 254 are predicted. The one or more parameters 274 may be generated using any suitable prediction process, such as, for example, a trained parameter prediction model 272. The trained parameter prediction model 272 may include any suitable framework, such as, for example, a trained gradient boosting model such as XGB, a trained tree-based model such as random forest and/or decision tree models, and/or any other suitable machine learning model. In some embodiments, the trained parameter prediction model 272 includes an XGB framework. Although specific embodiments are discussed herein, it will be appreciated that any suitable combination of machine learning frameworks may be implemented by the parameter prediction model 272.

The parameter prediction model 272 may be configured to receive one or more model inputs 270 and generate one or more parameter 274 predictions. In some embodiments, the one or more model inputs 270 includes one or more parameters or features related to a field of the prediction, the compliance event notification 256, and/or any other suitable input. For example, the model inputs 270 may include, but is not limited to, compliance-related features, demographic-related features, domain-specific features, product-related features, event-related features, etc. The model inputs 270 may include one or more inputs selected prior to model training and/or one or more inputs selected iteratively during a model training process, as discussed in greater detail below. The model inputs 270 may include a subset of the model inputs 270 and/or may share input values with the a subset of the model inputs 270 utilized by the integrated machine learning framework 268. It will be appreciated that any suitable set and/or subset of model inputs 270 may be provided to each of the trained models, e.g., the compliance prediction model 262, the integrated machine learning framework 268, the parameter prediction model 272, etc.

As one non-limiting example, in embodiments including a compliance object 254 representative of a medication regimen, the parameter prediction model 272 may be configured to predict a parameter of a prescription fill (e.g., a predicted compliance state corresponding to a fill or refill of a prescription). In some embodiments, the parameter prediction model 272 may be configured to predict an exhaustion date for the prescription fill (e.g., predict a number of days for the corresponding prescription fill). Although specific embodiments are discussed herein, it will be appreciated that any suitable parameter of a predicted next regimen compliance state 264 may be predicted by a parameter prediction model 272. As another non-limiting example, in embodiments including a compliance object 254 representative of a maintenance the parameter prediction model 272 may be configured to predict a parameter of a next maintenance period (e.g., next date on which maintenance will be required).

Although embodiments are illustrated herein including the compliance prediction model 262 and the integrated machine learning framework 268 as separate from the parameter prediction model 272, it will be appreciated that the parameter prediction model 272 may be integrated into the compliance prediction model 262 and/or the integrated machine learning framework 268. For example, in some embodiments, the compliance prediction model 262 may include a first integrated machine learning framework 268 integrated with a randomized simulation framework and configured to generate a first output, such as a next regimen compliance state 264, and a second machine learning framework configured to generate a second output, such as one or more predicted next regimen compliance state parameters. In some embodiments, execution of the second machine learning model may be dependent on the output of the first machine learning model and/or an output of a randomized simulation framework. For example, in some embodiments, the parameter prediction model 272 is invoked (e.g., executed) only when the compliance prediction model 262 and/or the integrated machine learning framework 268 predicts a compliant next regimen compliance state 264.

At step 214, a prediction output 276 is generated. The prediction output 276 includes data representative of a final predicted next regimen compliance state 264, the prediction period for the final predicted next regimen compliance state 264, a probability of the final predicted next regimen compliance state 264, one or more parameters of the final predicted next regimen compliance state 264, and/or any other suitable data. For example, in some embodiments, when the predicted next regimen compliance state 264 includes a compliant state, the prediction output 276 may include data indicating a predicted compliant state, the prediction time period during which the compliant state was predicted, and one or more parameters of the predicted compliant state generated by the parameter prediction model 272. As another example, in some embodiments, when the predicted next regimen compliance state 264 includes a non-compliant state, the prediction output 276 may include data indicating predicted non-compliance, the final prediction time period, and the probability of non-compliance for one or more predicted time periods. It will be appreciated that any suitable data related to the predicted next regimen compliance state 264 may be included in the prediction output.

At step 216, the predetermined regimen data structure 252 and/or the corresponding compliance object 254 is classified in one of a plurality of compliance classifications based, at least in part, on the prediction output 276. In some embodiments, a classification module 278 is configured to implement one or more classification processes, such as, for example, a trained classification model and/or a rules-based classification process, and output a classification 280. The number of predetermined categories may include any suitable number of categories corresponding to various confidence thresholds and/or simulation outputs, such as, for example, two categories, three categories, four categories, etc. A predetermined regimen data structure 252 may be categorized into one of the predetermined categories based on prediction thresholds (e.g., confidence levels, probability values, etc.) and/or predicted outcomes. For example, in some embodiments, the predetermined regimen data structure 252 may be classified into one of a plurality of classifications including compliant-high confidence, compliant-low confidence, non-compliant low-confidence, and non-compliant-high confidence based on the next regimen compliance state 264 and the probability included in the prediction output 276. The compliant/non-compliant classification may be based on the next regimen compliance state 264 (e.g., whether the next regimen compliance state 264 is a compliant or non-compliant state) and the low-confidence/high-confidence classification may be based on the probability of the next regimen compliance state 264.

As another example, in some embodiments, the classification 280 may correspond to a confidence level that the triggering compliance event will be remedied within a predetermined time period. For example, the predetermined classifications may include four classifications, a likely compliant category (indicating an expected compliance and a high confidence or probability for the prediction), a potentially compliant category (indicating expected compliance with lower confidence or probability for the prediction), a potentially non-compliant category (indicating an expected non-compliance but lower confidence of probability for the prediction), and a likely non-compliant category (indicating an expected non-compliance with a high confidence or probability for the prediction). Although specific embodiments are discussed herein, it will be appreciated that any suitable number of predetermined categories including any suitable labels and sorting criteria may be selected.

At step 218, an electronic compliance intervention data structure 282 may be generated and/or associated with a predetermined regimen data structure 252 based on the corresponding classification 280. For example, in some embodiments, a plurality of electronic intervention data structures 282 may be predefined. Each of the plurality of electronic intervention data structures 282 may initiate and/or automate generation and transmission of one or more electronic communications to a user device associated with a predetermined regimen data structure 252. A specific electronic intervention data structure 282 may be assigned to each predetermined regimen data structure 252 categorized in a specific classification 280, such as each predetermined regimen data structure 252 classified into a class indicating a non-compliant (e.g., probable, likely, etc.) outcome. The electronic compliance intervention data structure 282 for each classification 280 may be selected to provide an increase in a likelihood of compliance after one or more compliance interventions defined by the electronic compliance intervention data structure 282 have been executed.

In some embodiments, each predetermined classification may include a selected electronic compliance intervention data structure 282. For example, to continue the example above including four compliance categories, a likely compliant category may have a first electronic compliance intervention data structure 282 defining minimal (or no) compliance interventions, as it is likely that the predetermined regimen data structure 252 in the likely compliant category will be in compliance with a compliance event within an acceptable time period and without intervention. An electronic compliance intervention data structure 282 defining minimal (or no) compliance interventions allows compute resources to be preserved for application to higher impact interventions, such as those defined for other classifications of predetermined regimen data structures 252.

In some embodiments, one or more process-implemented rules may be applied to determine a classification for each electronic compliance intervention data structure 282 and/or to determine if an electronic compliance intervention data structure 282 is required. For example, in some embodiments, a first rule may be applied such that a predetermined regimen data structure 252 having a prior and predicted compliance behavior above a first predetermined threshold (e.g., above 95%, above 90%, etc.) does not require generation of a compliance intervention data structure 282, as the predetermined regimen data structure 252 will most likely be compliant at the end of the prediction period. Similarly, a second rule may be applied such that a predetermined regimen data structure 252 having a prior and/or predicted compliance behavior below a second predetermined threshold (e.g., below 50%, below 30%, etc.) does not require generation of an electronic compliance intervention data structure 282, as the predetermined regimen data structure 252 will most likely not be in compliance at the end of the prediction period no matter the number or type of electronic interventions implemented.

In some embodiments, the prior and/or predicted compliance behavior may be used to select one or more categories of electronic compliance intervention data structures 282. To continue to the foregoing example, a second electronic compliance intervention data structure 282 may be associated with predetermined regimen data structures 252 categorized as probably compliant and/or probably non-compliant. For example, an electronic compliance intervention data structure 282 defining low-resource compliance intervention, such as an automated email campaign, may be sufficient to shift predicted probably compliant and/or probably non-compliant predetermined regiment data structures 252 to a likely compliant category, and thus higher resource compliance interventions are not needed. Finally, a likely non-compliant category may have a third electronic compliance intervention data structure 282 defining high resource compliance interventions associated therewith, as the high probability of non-compliance necessitates additional resource expenditure to shift a prediction to a likely and/or probably compliant category. It will be appreciated that any level and/or type of compliance intervention may be associated with any of the predetermined categories.

As illustrated in FIG. 5, in some embodiments, an electronic compliance intervention data structure 282 may include a last day data element 259 corresponding to a determined last day of effective intervention. For example, electronic compliance interventions, such as electronic communications 284 automatically generated based on an electronic compliance intervention data structure 282, may each include an expected response time associated therewith. The expected response time represents the amount of time that is expected or required for a positive response to an electronic compliance communication after generation of the corresponding communication. In some embodiments, an electronic compliance intervention data structure 282 includes a last day data elements that indicates a last date by which the corresponding set of compliance interventions must be implemented in order to provide the required or expected time for a positive response resulting in compliance with the underlying regimen.

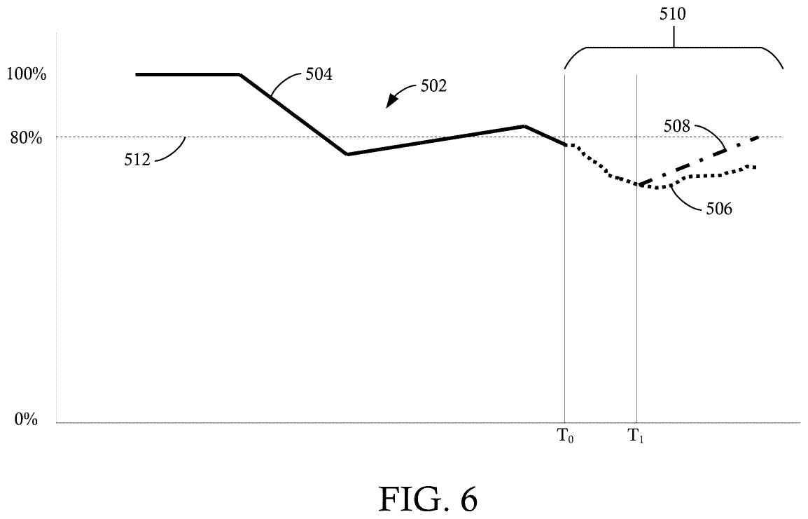

As one non-limiting example, FIG. 6 is a graph 500 illustrating a compliance trajectory 502 for a corresponding predetermined regimen data structure 252. The compliance trajectory 502 includes an actual behavior portion 504 representing the known or actual compliance behavior associated with the predetermined regimen data structure 252 and a predicted behavior portion 506 representing a predicted compliance behavior upon occurrence of a compliance event at time to. As illustrated in FIG. 6, the predicted behavior portion 506 indicates that the prediction output 276 predicts non-compliance of the corresponding predetermined regimen data structure 252 over the entirety of a prediction period 510. An electronic compliance intervention data structure 282 is generated including compliance intervention activities configured to drive compliance of the predetermined regimen data structure 252. A desired behavior portion 508 illustrates a slope of expected or desired behavior based on the compliance intervention activities of the electronic compliance intervention data structure 282. In order to provide adequate time to reach compliance prior to the end of the prediction period 510, e.g., to provide time for the desired behavior portion 508 to intersect a compliance threshold 512, the intervention activities within the electronic compliance intervention data structure 282 must be implemented by time t₁.

In some embodiments, a last day data element 259 may be used to determine strategic deployment of limited intervention resources. For example, certain electronic intervention resources, such as process resources, time resources, labor resources, etc. may have a limited availability for any given time period (e.g., each day, week, etc.). A last day data element 259 may be utilized to prioritize electronic compliance intervention data structures 282 for which an earlier intervention is required. Where a limited number of electronic intervention resources are available, electronic compliance intervention data structures 282 having an earlier last day data element 259 may be selected to utilize the available resources as compared to electronic compliance intervention data structures 282 having later last day data elements 259, as later interventions may still be effective for the later electronic compliance intervention data structures 282. It will be appreciated that any suitable prioritization process may be applied to select electronic compliance intervention data structures 282 for execution and/or implementation.

At step 220, feedback data 290 may be received and one or more updated models may be generated. Feedback data 290 may include data representative of results and/or outcomes related to the compliance event notification 256 and/or the electronic compliance intervention data structure 282. For example, in some embodiments, feedback data 290 may include actual state data for the compliance object 254 for an elapsed time period at least partially overlapping with an aggregated time period defined by the prediction time periods (e.g., actual state for a given prediction time period, state parameters for an actual state, etc.). As another example, in some embodiments, feedback data 290 may include state predictions generated by a separate prediction framework. It will be appreciated that any suitable feedback data 290 may be received.

One or more updated models may be generated for use in subsequent execution of the compliance intervention determination engine 258. Updated models may be generated by any suitable system, method, engine, etc., such as, for example, a model generation engine 292. In some embodiments, an updated training dataset may be generated by combining, augmenting, and/or modifying a prior training dataset based on the feedback data 290. For example, in some embodiments, an updated training dataset may be generated by adding additional labeled data representative of actual states observed for one or more predicted states having a corresponding actual state in the feedback data 290.

At step 222, a prediction interface may be generated. The prediction interface may include one or more interface elements configured to display and/or otherwise present outcomes of the randomized simulations and/or enable execution of one or more selected electronic compliance intervention data structures 282. For example, in some embodiments, a matrix interface element may be generated including prediction values and feature values and/or any other applicable values for determining the prediction values. As another example, in some embodiments, a model interface element may be generated that includes metrics related to execution of the integrated machine learning framework.

In some embodiments, the prediction interface may include interface elements related to the predicted next regimen compliance state(s) 264 and interface elements related to actual next regimen compliance states of the compliance object 254. For example, in some embodiments, at least a portion of the prediction time periods applied during steps 204 and 206 may overlap with time periods that have elapsed in real time (e.g., a prediction time period including one or more days of a month (e.g., September 2023) that is a prior time period at the time the prediction interface is generated (e.g., in October 2023). In such embodiments, the actual states for one or more prediction time period and/or one or more aggregated prediction time periods may be provided in one or more interface elements in conjunction with the predicted next regimen compliance state 264 for the corresponding time periods.

As one non-limiting example, in some embodiments, a prediction interface may include interface elements displaying aggregated non-compliant prediction states for an aggregated time period (e.g., one week time periods where the prediction period was defined in days) and actual states for the corresponding aggregated time periods (e.g., compliant if the compliance object 254 was in a compliant state during any portion of the aggregated time period and non-compliant otherwise). As another non-limiting example, in some embodiments, a prediction interface may include elements displaying parameters of a predicted compliant state (e.g., predicted fill amount for a corresponding medication regimen) and parameters of an actual compliant state (e.g., actual fill amount for a corresponding medication regimen). It will be appreciated that any suitable interface elements may be provided to display actual and/or predicted states and/or state elements.

It will be appreciated that next regimen compliance state determinations and/or next regimen compliance state parameter predictions as disclosed herein, particularly on large datasets intended to be used with healthcare regimens, maintenance regimens, etc., is only possible with the aid of computer-assisted machine-learning algorithms and techniques, such as the disclosed compliance prediction model 262 including the integrated machine learning framework 268 and/or the parameter prediction model 272. In some embodiments, machine learning processes including prediction frameworks, such as XGB and/or tree-based frameworks, are used to perform operations that cannot practically be performed by a human, either mentally or with assistance, such as predicting a next regimen compliance state 264 and/or parameters of a next regimen compliance state 264. It will be appreciated that a variety of machine learning techniques can be used alone or in combination to generate trained compliance prediction models 262 and/or trained parameter prediction models 272.

As one non-limiting example, the automated intervention generation method 200 may be implemented for automatically generating electric compliance interventions related to a medication regimen (e.g., a prescription regimen, a vitamin regimen, etc.). When a medication regimen is prescribed (e.g., when a doctor or other healthcare professional prescribes a medication to a patient), a predetermined regimen data structure 252 may be generated in a database, such as database 14. The predetermined regimen data structure 252 includes a compliance object 254 including data elements defining at least a portion of the medication regimen. For example, in some embodiments, the compliance object 254 may include data representative of the prescription period (e.g., on-going, thirty days, etc.), the initial fill period (e.g., thirty days, sixty days, etc.), the date of the initial fill, etc. It will be appreciated that the predetermined regimen data structures 252 may be generated and/or stored in compliance with any suitable requirements, such as, for example, HIPAA requirements.

A compliance event notification 256, such as a simulated compliance event notification 256, may be generated and/or received indicating exhaustion of the initial prescription fill. For example, a compliance event notification 256 may be simulated prior to the exhaustion date of the initial prescription fill to predict compliance with the medication regimen and to determine what, if any, electronic intervention communications should be generated. A next regimen compliance state 264 is predicted indicating whether the corresponding prescription is predicted to be refilled on an initial date after the exhaustion of the initial prescription fill. For example, an initial prediction time period may include the exhaustion date. At step 204 of the method, a predicted next regimen compliance state 264 for the initial prediction time period (e.g., Day₀) is generated. If the predicted next regimen compliance state 264 indicates a non-compliant state (e.g., the predicted next regimen compliance state 264 predicts that the prescription will not be filled on day Day₀, the prediction time period is incremented by an increment amount (e.g., one day), and a next regimen compliance state is predicted for the updated prediction time period (e.g., Day₁). The prediction time period may be incremented until a prescription fill is predicted (e.g., a compliant state is predicted) or a maximum number of predictions have been generated (e.g., a final prediction period (e.g., Day_{Final}) has been calculated).

If a compliant state is predicted (e.g., a prescription fill is predicted), a parameter of the compliant state, such as a number of days for the prescription fill, is predicted. For example, in some embodiments, a prescription fill may be obtained for a variable number of days, such as 30 days, 60 days, 90 days, etc. When a prescription fill is predicted, for example by a compliance prediction model 262 including an integrated machine learning framework 268, a parameter prediction model 272 may be configured to predict the number of days for the prescription fill based on one or more inputs.

A prediction interface may be generated including a final predicted state (e.g., a prescription fill during one of the prediction time periods or no prescription fill during a final prediction time period). A matrix interface element may be configured to display the prediction time periods, predicted states, and/or prediction confidence levels. In some embodiments, a graphical interface element may be configured to provide a graphical representation of the predicted states, prediction time periods, and/or prediction confidence levels.

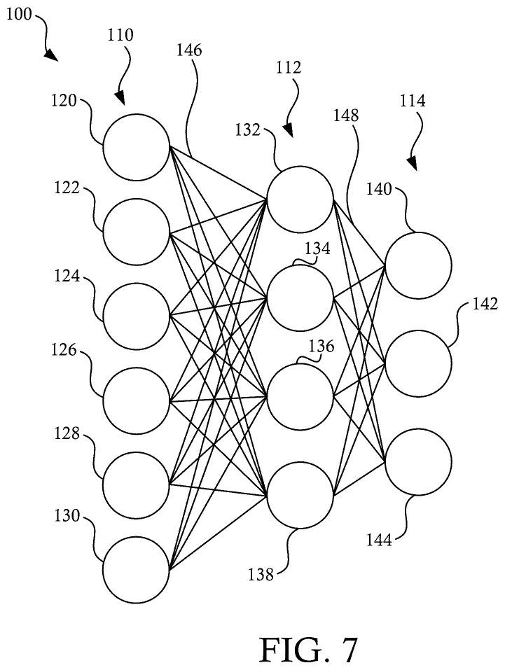

FIG. 7 illustrates an artificial neural network 100, in accordance with some embodiments. Alternative terms for “artificial neural network” are “neural network,” “artificial neural net,” “neural net,” or “trained function.” The neural network 100 comprises nodes 120-144 and edges 146-148, wherein each edge 146-148 is a directed connection from a first node 120-138 to a second node 132-144. In general, the first node 120-138 and the second node 132-144 are different nodes, although it is also possible that the first node 120-138 and the second node 132-144 are identical. For example, in FIG. 7 the edge 146 is a directed connection from the node 120 to the node 132, and the edge 148 is a directed connection from the node 132 to the node 140. An edge 146-148 from a first node 120-138 to a second node 132-144 is also denoted as “ingoing edge” for the second node 132-144 and as “outgoing edge” for the first node 120-138.

The nodes 120-144 of the neural network 100 may be arranged in layers 110-114, wherein the layers may comprise an intrinsic order introduced by the edges 146-148 between the nodes 120-144 such that edges 146-148 exist only between neighboring layers of nodes. In the illustrated embodiment, there is an input layer 110 comprising only nodes 120-130 without an incoming edge, an output layer 114 comprising only nodes 140-144 without outgoing edges, and a hidden layer 112 in-between the input layer 110 and the output layer 114. In general, the number of hidden layer 112 may be chosen arbitrarily and/or through training. The number of nodes 120-130 within the input layer 110 usually relates to the number of input values of the neural network, and the number of nodes 140-144 within the output layer 114 usually relates to the number of output values of the neural network.

In particular, a (real) number may be assigned as a value to every node 120-144 of the neural network 100. Here, xᵢ⁽ⁿ⁾ denotes the value of the i-th node 120-144 of the n-th layer 110-114. The values of the nodes 120-130 of the input layer 110 are equivalent to the input values of the neural network 100, the values of the nodes 140-144 of the output layer 114 are equivalent to the output value of the neural network 100. Furthermore, each edge 146-148 may comprise a weight being a real number, in particular, the weight is a real number within the interval [−1, 1], within the interval [0, 1], and/or within any other suitable interval. Here, w_{i,j}^{(m,n)} denotes the weight of the edge between the i-th node 120-138 of the m-th layer 110, 112 and the j-th node 132-144 of the n-th layer 112, 114. Furthermore, the abbreviation w_{i,j}⁽ⁿ⁾ is defined for the weight w_{i,j}^{(n,n+1)}.

In particular, to calculate the output values of the neural network 100, the input values are propagated through the neural network. In particular, the values of the nodes 132-144 of the (n+1)-th layer 112, 114 may be calculated based on the values of the nodes 120-138 of the n-th layer 110, 112 by

$${x}_{j}^{( n + 1 )} = f ( {\sum }_{i} {x}_{i}^{( n )} \cdot {w}_{i , j}^{( n )} )$$

Herein, the function f is a transfer function (another term is “activation function”). Known transfer functions are step functions, sigmoid function (e.g., the logistic function, the generalized logistic function, the hyperbolic tangent, the Arctangent function, the error function, the smooth step function) or rectifier functions. The transfer function is mainly used for normalization purposes.

In particular, the values are propagated layer-wise through the neural network, wherein values of the input layer 110 are given by the input of the neural network 100, wherein values of the hidden layer(s) 112 may be calculated based on the values of the input layer 110 of the neural network and/or based on the values of a prior hidden layer, etc.

In order to set the values w_{i,j}^{(m,n)} for the edges, the neural network 100 has to be trained using training data. In particular, training data comprises training input data and training output data. For a training step, the neural network 100 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 100 (backpropagation algorithm). In particular, the weights are changed according to

$${w}_{i , j}^{′ ( n )} = {w}_{i , j}^{( n )} - γ \cdot {δ}_{j}^{( n )} \cdot {x}_{i}^{( n )}$$

wherein γ is a learning rate, and the numbers δⱼ⁽ⁿ⁾ may be recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

based on δⱼ⁽ⁿ⁺¹⁾, if the (n+1)-th layer is not the output layer, and

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)-th layer is the output layer 114, wherein f is the first derivative of the activation function, and yⱼ⁽ⁿ⁺¹⁾ is the comparison training value for the j-th node of the output layer 114.

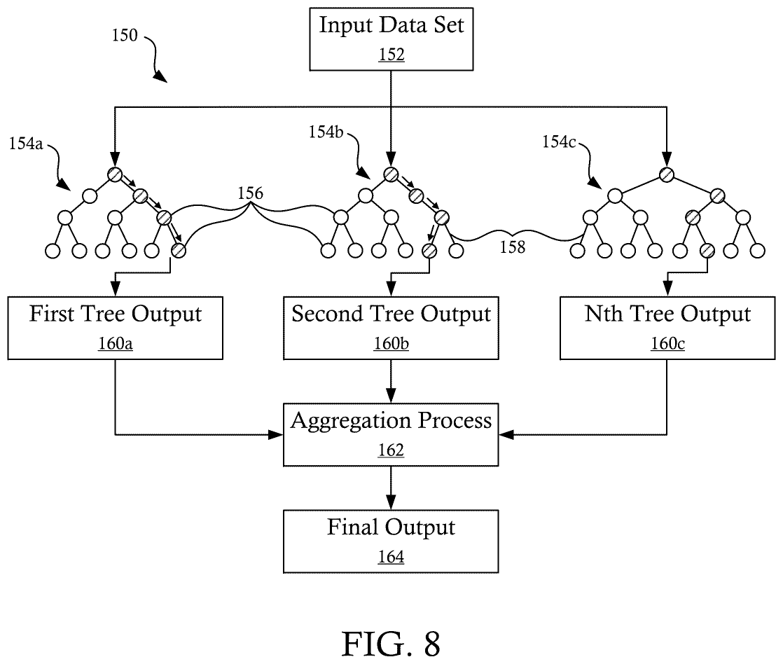

FIG. 8 illustrates a tree-based neural network 150, in accordance with some embodiments. In particular, the tree-based neural network 150 is a random forest neural network, though it will be appreciated that the discussion herein is applicable to other decision tree neural networks. The tree-based neural network 150 includes a plurality of trained decision trees 154a-154c each including a set of nodes 156 (also referred to as “leaves”) and a set of edges 158 (also referred to as “branches”).

Each of the trained decision trees 154a-154c may include a classification and/or a regression tree (CART). Classification trees include a tree model in which a target variable may take a discrete set of values, e.g., may be classified as one of a set of values. In classification trees, each leaf 156 represents class labels and each of the branches 158 represents conjunctions of features that connect the class labels. Regression trees include a tree model in which the target variable may take continuous values (e.g., a real number value).

In operation, an input data set 152 including one or more features or attributes is received. A subset of the input data set 152 is provided to each of the trained decision trees 154a-154c. The subset may include a portion of and/or all of the features or attributes included in the input data set 152. Each of the trained decision trees 154a-154c is trained to receive the subset of the input data set 152 and generate a tree output value 160a-160c, such as a classification or regression output. The individual tree output value 160a-160c is determined by traversing the trained decision trees 154a-154c to arrive at a final leaf (or node) 156.

In some embodiments, the tree-based neural network 150 applies an aggregation process 162 to combine the output of each of the trained decision trees 154a-154c into a final output 164. For example, in embodiments including classification trees, the tree-based neural network 150 may apply a majority-voting process to identify a classification selected by the majority of the trained decision trees 154a-154c. As another example, in embodiments including regression trees, the tree-based neural network 150 may apply an average, mean, and/or other mathematical process to generate a composite output of the trained decision trees. The final output 164 is provided as an output of the tree-based neural network 150.

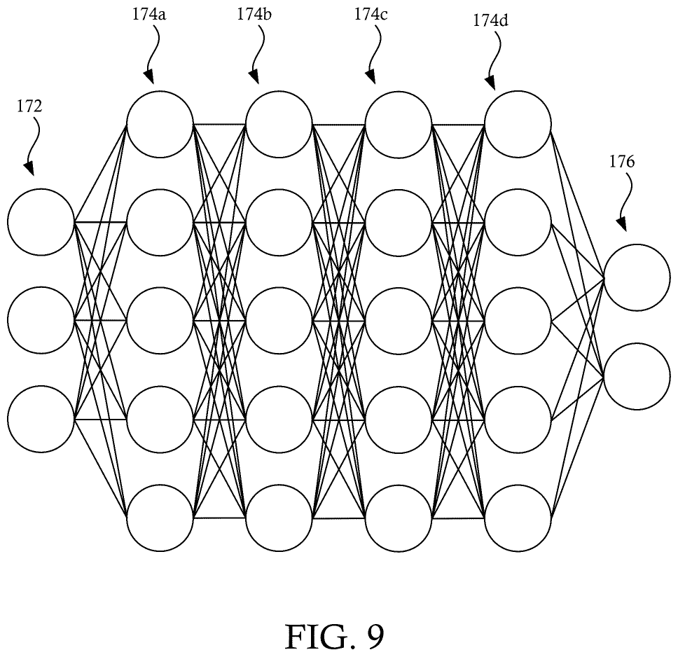

FIG. 9 illustrates a deep neural network (DNN) 170, in accordance with some embodiments. The DNN 170 is an artificial neural network, such as the neural network 100 illustrated in conjunction with FIG. 3, that includes representation learning. The DNN 170 may include an unbounded number of (e.g., two or more) intermediate layers 174a-174d each of a bounded size (e.g., having a predetermined number of nodes), providing for practical application and optimized implementation of a universal classifier. Each of the layers 174a-174d may be heterogenous. The DNN 170 may be configured to model complex, non-linear relationships. Intermediate layers, such as intermediate layer 174c, may provide compositions of features from lower layers, such as layers 174a, 174b, providing for modeling of complex data.

In some embodiments, the DNN 170 may be considered a stacked neural network including multiple layers each configured to execute one or more computations. The computation for a network with L hidden layers may be denoted as:

$$f ( x ) = f [ {a}^{( L + 1 )} ( {h}^{( L )} ( {a}^{( L )} ( … ( {h}^{( 2 )} ( {a}^{( 2 )} ( {h}^{( 1 )} ( {a}^{( 1 )} ( x ) ) ) ) ) ) ) ) ]$$

where a^{(l)}(x) is a preactivation function and h^{(l)}(x) is a hidden-layer activation function providing the output of each hidden layer. The preactivation function a^{(l)}(x) may include a linear operation with matrix W^{(l)} and bias b^{(l)}, where:

$${a}^{( l )} ( x ) = {W}^{( l )} x + {b}^{( l )}$$

In some embodiments, the DNN 170 is a feedforward network in which data flows from an input layer 172 to an output layer 176 without looping back through any layers. In some embodiments, the DNN 170 may include a backpropagation network in which the output of at least one hidden layer is provided, e.g., propagated, to a prior hidden layer. The DNN 170 may include any suitable neural network, such as a self-organizing neural network, a recurrent neural network, a convolutional neural network, a modular neural network, and/or any other suitable neural network.

In some embodiments, a DNN 170 may include a neural additive model (NAM). An NAM includes a linear combination of networks, each of which attends to (e.g., provides a calculation regarding) a single input feature. For example, a NAM may be represented as:

$$y = β + {f}_{1} ( {x}_{1} ) + {f}_{2} ( {x}_{2} ) + … + {f}_{K} ( {x}_{K} )$$

where β is an offset and each fᵢ is parametrized by a neural network. In some embodiments, the DNN 170 may include a neural multiplicative model (NMM), including a multiplicative form for the NAM mode using a log transformation of the dependent variable y and the independent variable x:

$$y = {e}^{β} {e}^{f ( log x )} {e}^{{Σ}_{i} {f}_{i}^{d} ( {d}_{i} )}$$

where d represents one or more features of the independent variable x.

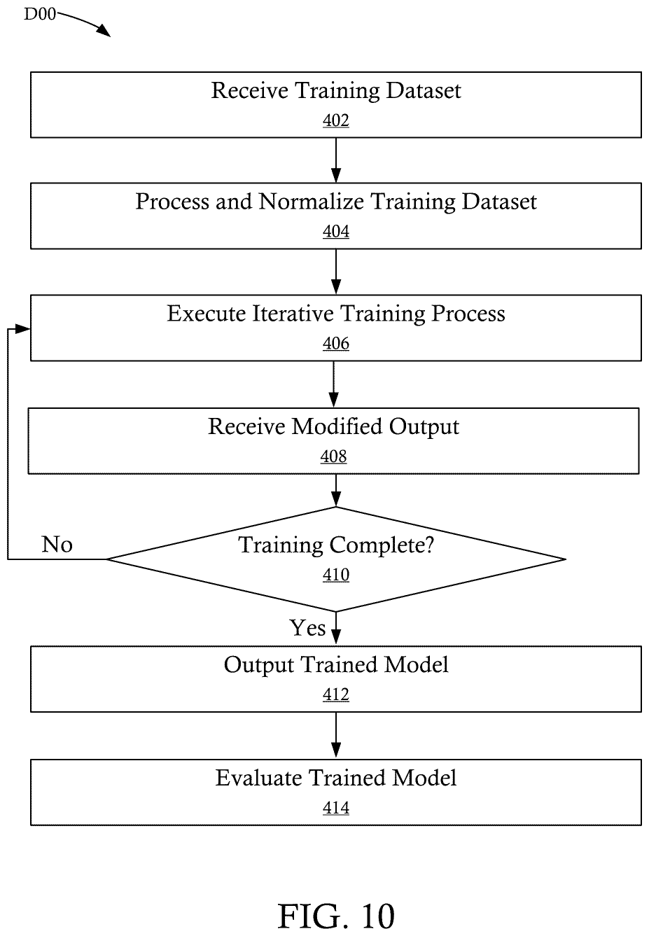

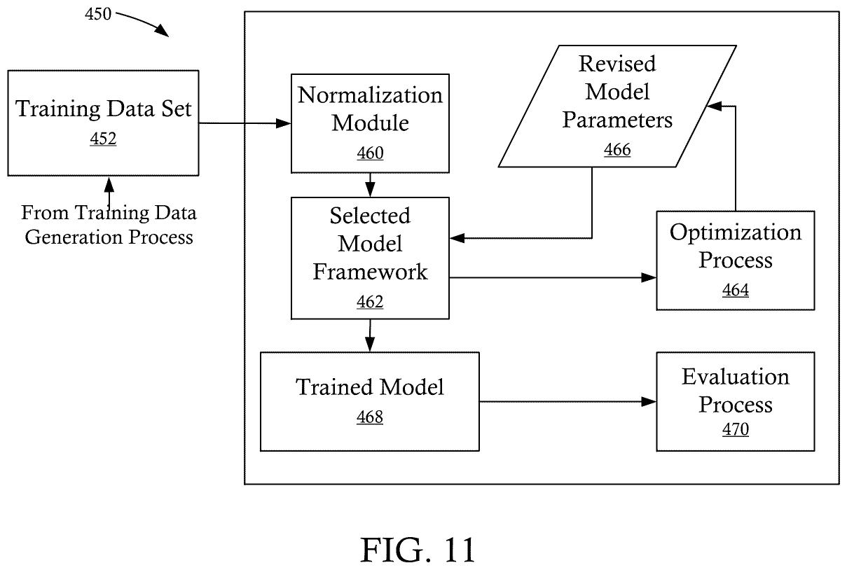

In some embodiments, a compliance prediction model and/or a parameter prediction model can include and/or implement one or more trained models, such as a one or more trained XGB models, tree models, boost models, etc. In some embodiments, one or more trained models can be generated using an iterative training process based on a training dataset. FIG. 10 illustrates a method 400 for generating a trained model, such as a trained optimization model, in accordance with some embodiments. FIG. 11 is a process flow 450 illustrating various steps of the method 400 of generating a trained model, in accordance with some embodiments. At step 402, a training dataset 452 is received by a system, such as a processing device 10. The training dataset 452 can include labeled and/or unlabeled data. For example, in some embodiments, a set of labeled and/or semi-labeled data is provided for use in training a model. In some embodiments, the training dataset 452 includes prior regimen compliance data for prior time periods and/or feedback data from predicted regimen compliance periods.

At optional step 404, the received training dataset 452 is processed and/or normalized by a normalization module 460. For example, in some embodiments, the training dataset 452 can be augmented by imputing or estimating missing values of one or more features associated with regimen compliance. In some embodiments, processing of the received training dataset 452 includes outlier detection configured to remove data likely to skew training of a compliance prediction and/or parameter prediction model. In some embodiments, processing of the received training dataset 452 includes removing features that have limited value with respect to training of the respective models.

At step 406, an iterative training process is executed to train a selected model framework 462. The selected model framework 462 can include an untrained (e.g., base) machine learning model, such as an XGB model, a random forest model, a decision tree model, and/or a partially or previously trained model (e.g., a prior version of a trained model). The training process is configured to iteratively adjust parameters (e.g., hyperparameters) of the selected model framework 462 to minimize a cost value (e.g., an output of a cost function) for the selected model framework 462. In some embodiments, the cost value is related to a difference between predicted and actual behavior with respect to regimen compliance.

The training process is an iterative process that generates set of revised model parameters 466 during each iteration. The set of revised model parameters 466 can be generated by applying an optimization process 464 to the cost function of the selected model framework 462. The optimization process 464 can be configured to reduce the cost value (e.g., reduce the output of the cost function) at each step by adjusting one or more parameters during each iteration of the training process.

After each iteration of the training process, at step 408, a determination is made whether the training process is complete. The determination at step 408 can be based on any suitable parameters. For example, in some embodiments, a training process can complete after a predetermined number of iterations. As another example, in some embodiments, a training process can complete when it is determined that the cost function of the selected model framework 462 has reached a minimum, such as a local minimum and/or a global minimum.

At step 410, a trained model 468, such as a trained compliance prediction model or a trained parameter prediction model, is output and provided for use in an automated intervention process, such as the automated intervention generation method 200 discussed above with respect to FIGS. 3-5. At optional step 412, a trained model 468 can be evaluated by an evaluation process 470. A trained model can be evaluated based on any suitable metrics, such as, for example, an F or F1 score, normalized discounted cumulative gain (NDCG) of the model, mean reciprocal rank (MRR), mean average precision (MAP) score of the model, and/or any other suitable evaluation metrics. Although specific embodiments are discussed herein, it will be appreciated that any suitable set of evaluation metrics can be used to evaluate a trained model.

Although the subject matter has been described in terms of exemplary embodiments, it is not limited thereto. Rather, the appended claims should be construed broadly, to include other variants and embodiments, which may be made by those skilled in the art.

What is claimed is:

1. A system, comprising:
a non-transitory memory;
a processor communicatively coupled to the non-transitory memory, wherein the processor is configured to read a set of instructions to:
receive a compliance event notification related to a regimen data structure;
iteratively predict a next regimen compliance state for the regimen data structure for a prediction time period, wherein the next regimen compliance state is predicted by a randomized prediction model including an integrated machine learning framework, and wherein the prediction time period is incremented by a predetermined increment during each iteration;
when the next regimen compliance state comprises a compliant state, predict at least one parameter of the next regimen compliance state by implementing a trained parameter prediction model comprising a tree-based machine learning framework;
select an intervention communication data structure based on the next regimen compliance state; and
modify the regimen data structure to reference the intervention communication data structure.

2. The system of claim 1, wherein the randomized prediction model comprises a Monte Carlo framework.

3. The system of claim 1, wherein the integrated machine learning framework comprises an extreme gradient boost framework.

4. The system of claim 1, wherein the next regimen compliance state is iteratively predicted until a predetermined number of increments have been performed or a compliant state is predicted.

5. The system of claim 1, wherein the processor is configured to read the set of instructions to, prior to selecting the intervention communication data structure, classify the regimen data structure in one of a plurality of categories based on the next regimen compliance state and a probability of the next regimen compliance state, wherein the intervention communication data structure is selected based on the one of the plurality of categories.

6. The system of claim 5, wherein the plurality of categories comprise a likely compliant category associated with a compliant next regimen compliance state and a probability above a first predetermined threshold, a potentially compliant category associated with the compliant next regimen compliance state and a probability below the first predetermined threshold, a potentially non-compliant category associated with a non-compliant next regimen compliance state and a probability below a second predetermined threshold, and a likely non-compliant category associated with the non-compliant next regimen compliance state and a probability above the second predetermined threshold.

7. The system of claim 1, wherein the regimen data structure includes data representative of a medication regimen.

8. A computer-implemented method, comprising:
receiving a compliance event notification related to a regimen data structure;
iteratively predicting a next regimen compliance state for the regimen data structure for a prediction time period, wherein the next regimen compliance state is predicted by a randomized prediction model comprising a Monte Carlo framework including an integrated machine learning framework comprising an extreme gradient boost framework, and wherein the prediction time period is incremented by a predetermined increment during each iteration;
selecting an intervention communication data structure based on the next regimen compliance state; and
modifying the regimen data structure to reference the intervention communication data structure.

9. The computer-implemented method of claim 8, comprising, when the next regimen compliance state comprises a compliant state, predicting at least one parameter of the next regimen compliance state by implementing a trained parameter prediction model.

10. The computer-implemented method of claim 9, wherein the trained parameter prediction model comprises a tree-based machine learning framework.

11. The computer-implemented method of claim 8, wherein the next regimen compliance state is iteratively predicted until a predetermined number of increments have been performed or a compliant state is predicted.

12. The computer-implemented method of claim 8, comprising prior to selecting the intervention communication data structure, classifying the regimen data structure in one of a plurality of categories based on the next regimen compliance state and a probability of the next regimen compliance state, wherein the intervention communication data structure is selected based on the one of the plurality of categories.

13. The computer-implemented method of claim 12, wherein the plurality of categories comprise a likely compliant category associated with a compliant next regimen compliance state and a probability above a first predetermined threshold, a potentially compliant category associated with the compliant next regimen compliance state and a probability below the first predetermined threshold, a potentially non-compliant category associated with a non-compliant next regimen compliance state and a probability below a second predetermined threshold, and a likely non-compliant category associated with the non-compliant next regimen compliance state and a probability above the second predetermined threshold.

14. A non-transitory computer readable medium having instructions stored thereon, wherein the instructions, when executed by at least one processor, cause at least one device to perform operations comprising:
receiving a compliance event notification related to a regimen data structure;
iteratively predicting a next regimen compliance state for the regimen data structure for a prediction time period, wherein the next regimen compliance state is predicted by a randomized prediction model including an integrated machine learning framework, and wherein the prediction time period is incremented by a predetermined increment during each iteration;
classifying the regimen data structure in one of a plurality of categories based on the next regimen compliance state and a probability of the next regimen compliance state;
when the next regimen compliance state comprises a compliant state, predicting at least one parameter of the next regimen compliance state by implementing a trained parameter prediction model comprising a tree-based machine learning framework;
selecting an intervention communication data structure based on the one of the plurality of categories; and
modifying the regimen data structure to reference the intervention communication data structure.

15. The non-transitory computer readable medium of claim 14, wherein the randomized prediction model comprises a Monte Carlo framework.

16. The non-transitory computer readable medium of claim 15, wherein the integrated machine learning framework comprises an extreme gradient boost framework.

17. The non-transitory computer readable medium of claim 15, wherein the plurality of categories comprise a likely compliant category associated with a compliant next regimen compliance state and a probability above a first predetermined threshold, a potentially compliant category associated with the compliant next regimen compliance state and a probability below the first predetermined threshold, a potentially non-compliant category associated with a non-compliant next regimen compliance state and a probability below a second predetermined threshold, and a likely non-compliant category associated with the non-compliant next regimen compliance state and a probability above the second predetermined threshold.

18. The non-transitory computer readable medium of claim 14, wherein the next regimen compliance state is iteratively predicted until a predetermined number of increments have been performed or a compliant state is predicted.

19. The non-transitory computer readable medium of claim 14, wherein the processor is configured to read the set of instructions to, prior to selecting the intervention communication data structure, classify the regimen data structure in one of a plurality of categories based on the next regimen compliance state and a probability of the next regimen compliance state, wherein the intervention communication data structure is selected based on the one of the plurality of categories.

20. The non-transitory computer readable medium of claim 14, wherein the randomized prediction model comprises a Monte Carlo framework and the integrated machine learning framework comprises an extreme gradient boost framework.
